(19) United States
(12) Patent Application Publication
(10) Pub. No.: US 2003/0118520 A1
(43) Pub. Date: Jun. 26, 2003

(54) Cleaning composition for dental prostheses

(76) Inventors: Gerd Reinhardt, Kelkheim (DE); Torsten Henning, Bad Soden (DE); Wiebke Johannpeter, Steinbach (DE)

Correspondence Address: CLARIANT CORPORATION, INTELLECTUAL PROPERTY DEPARTMENT, 4000 MONROE ROAD, CHARLOTTE, NC, 28205, US

(21) Appl. No.: 10/338,407

(22) Filed: Jan. 8, 2003

Related U.S. Application Data
(63) Continuation of application No. 10/003,488, filed on Nov. 1, 2001.

(30) Foreign Application Priority Data
Nov. 3, 2000 (DE) 100 54 693.5

(51) Int. Cl. A61K 7/20

(52) U.S. Cl. 424/53

(57) ABSTRACT

Cleaning composition for dental prostheses comprising an oxidizing agent and an organic compound which carries a carbonyl group which forms a dioxirane structure in aqueous solution by reaction with the oxidizing agent.

### FIELD OF THE INVENTION

The invention relates to a cleaning composition in pulverulent or tablet form for the automatic cleaning and disinfection of dental prostheses in aqueous solution.

### BACKGROUND OF THE INVENTION

Cleaning compositions for dental prostheses are used by placing them in water at the same time as the dental prosthesis to be cleaned, as a result of which the cleaning compositions then disintegrate with the evolution of a greater or lesser amount of gas and release active ingredients which attack and remove the films of soil adhering to the dental prosthesis.

Cleaning compositions of this type are described, for example, in DE-23 12 847, 25 48 469, 26 58 450 and 27 41 072, in EP-00 10 412, 00 28 005 and 0102 418, and in EP-01 33 354, 0149 308, 0157 464 and 0225 658. The cleaning compositions essentially comprise oxygen-releasing substances for bleaching, oxidation and disinfection, inorganic softeners and lime-binding agents, chelating agents, gas-forming carriers, such as carbonates and/or bicarbonates, citric acid, tartaric acid, amidosulfonic acid or salts thereof, disinfectants such as benzoates and/or hydroxybenzoic alkyl esters, and lubricants, binders and/or disintegrants, surfactants, dye additives and, finally, aroma substance.

Upon dissolution in water, the pH values vary, depending on the composition, between acidic, neutral and alkaline salts. The cleaning compositions are mostly rapidly cleaning compositions; i.e. they have a cleaning time of from 10 to 15 minutes. In some cases, however, the cleaning compositions are products which act slowly and have relatively long use times. The complete cleaning action for a dental prosthesis to be treated will be accomplished in approximately 1 to 2 hours.

A disadvantage of these cleaning compositions is their low cleaning and bleaching action in the temperature range below 60° C. For this reason, EP-02 53 772 describes a cleaning tablet which additionally comprises a ! bleach activator, such as tetraacetylethylenediamine (TAED) in concentrations of from 5 to 15% by weight. The bleach activator used in the cleaning tablets initiates a reaction mechanism in aqueous solution where, as result of the reaction of the activator with perhydroxyl anions, peracetic acid is formed as intermediate which has strongly bleaching and disinfecting properties.

### SUMMARY OF THE INVENTION

Surprisingly, it has now been found that the effectiveness of cleaning compositions for dental prostheses can be considerably increased if a carbonyl-function-containing organic substance is added which is able to form an intermediate dioxirane structure with the oxidizing agent present.

The invention therefore provides cleaning compositions for dental prostheses, comprising at least one organic or inorganic oxidizing agent and at least one carbonyl-group-containing organic substance which forms a dioxirane structure in aqueous solution by reaction with oxidizing agents.

In this connection, a dioxirane structure is understood as meaning a three-membered ring consisting of a carbon atom and two oxygen atoms. As is known, dioxiranes can be obtained by reacting peroxornonosulfonic acids or salts thereof with certain carbonyl-group-carrying organic compounds. Dioxiranes then transfer an oxygen atom to the soil adhering to the dental prosthesis. After the bleaching process has taken place, the carbonyl compound reforms and is available for a new cycle. In this connection, it is advantageous that the carbonyl component only has to be used in low concentrations since it is, at least in the ideal case, catalytically active.

### DETAILED DESCRIPTION OF THE INVENTION

The use of dioxiranes in preparative organic chemistry has been known for some years. Examples thereof are described in Bull. Soc. Chem. Belg., Vol. 105, Volume 10-11, pp. 581-597 and in the literature cited therein. It is also known that mixtures of peroxymonosulfonic acids and certain carbonyl compounds can be used for bleaching textiles and hard surfaces. Examples thereof are listed in U.S. Pat. No. 3,822,114 and WO99/23197. However, it is not clear from any of the said publications that dioxiranes, prepared by reacting peroxysulfonic acids or salts thereof and certain carbonyl compounds, have excellent effectiveness for the cleaning of dental prostheses.

The invention further provides a method of bleaching and disinfecting dental prostheses, which comprises treating the dental prosthesis with an aqueous solution of a cleaning composition which comprises at least one organic or inorganic oxidizing agent and at least one carbonyl-group-containing organic substance which forms a dioxirane structure in aqueous solution by reaction with the oxidizing agent.

Suitable carbonyl-group-containing organic substances are preferably compounds of the formula

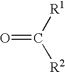

which, by reacting with the oxidizing agents, give the dioxirane compounds of the formula

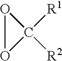

wherein R¹ and R are C₁-C₂₄-alkyl, C₂-C₂₄-akenyl or aryl groups which may be substituted by halogen atoms, such as F, Cl, Br or I, nitro, cyano or amino groups or groups of the formulae

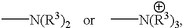

where R³ is C₁-C₄-alkyl, benzyl or phenyl. The radicals R¹ and R² may also be constituents of one and the same ring system. Particular preference is given here to ring systems in which R₁ and R₂ have a total of 4-7 carbon atoms. Such cyclic compounds in which R₁ and R₂ are constituents of a ring system may be further substituted.

Particularly preferred compounds of the carbonyl structure are: dimethyl ketone, methyl ethyl ketone, 1,1,1-trifluoroacetone, perfluoroacetone, diphenyl ketone, alkyl phenyl ketone, dipyridyl ketone, dipyridyl ketone N-oxide. From the group of the cyclic ketones, preference is given to: cyclopentanone, cyclohexanone, cycloheptanone, cyclooctanone and methyl derivatives thereof, and heterocyclic ketones, such as, for example N-acetyl-4-piperidone.

Particular preference is likewise given to amine-substituted ketones of the general formulae (III) and (IV) and the analogous ammonium-substituted compounds of the formulae (V) and (VI)

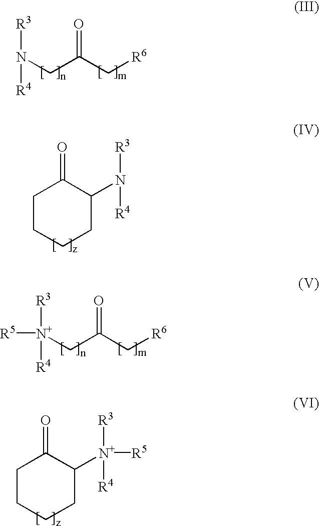

where R³ R⁴, R⁵ and R⁶ are C₁-C₂₂-alkyl, C₂-C₂₂-alkenyl or aryl. n=1-11, preferably 1-5, in particular 1-2, m=0-12, preferably 0-3, z=0,1, 2.

The positively charged ammonium ketones can contain negatively charged counterions to balance the charge from the group of halides, such as Cl⁻, Br⁻ or ROSO₃⁻, TosO⁻, SO₄²⁻ or CO₃²⁻, where R is C₁-C₄-alkyl and Tos is tosylate.

Examples of such compounds of the formulae III to VI are N,N-dimethylaminoacetone, N,N-diethylaminoacetone, N,N-dipropylaminoacetone, N,N-dibutylaminoacetone, N,N-dihexylaminoacetone, N,N-dioctylaminoacetone, N,N-didecylaminoacetone, N,N-didodecylaminoacetone, N,N-sterylaminoacetone, N-methyl-N-ethylaminoacetone, N-methyl-N-propylaminoacetone and N-methyl-N-butylaminoacetone, 2-(N,N-dimethylamino)cyclohexanone or 4-(N,N-dimethylamino)cyclohexanone and higher homologs thereof.

The carbonyl-group-containing compounds are used in the cleaning compositions according to the invention in concentrations of 0.01-10%, preferably 0.1-8% and in particular 0.5-5%.

Suitable inorganic oxidizing agents based on oxygen are primarily all alkali metal or ammonium peroxosulfates, such as, for example, potassium peroxomonosulfate (technically: Caroat® or Oxone®). In addition, it is, however, also possible to use alkali metal perborate mono- or tetrahydrates and/or alkali metal percarbonates, sodium being the preferred alkali metal. In a particularly preferred embodiment, mixtures of peroxysulfates with perborates or percarbonates in the mixing ratio 1:10 to 10:1, preferably 1:5 to 5:1 are used. In addition or alternatively, the cleaning compositions according to the invention can comprise organic-based oxidizing agents in a concentration range from 1-20%. These include all known peroxycarboxylic acids, e.g. monoperoxyphthalic acid, dodecanediperoxy acid or phthalimidoperoxycarboxylic acids (PAP). The concentration of the oxidizing agents in the cleaning compositions according to the invention is 5-90%, preferably 10-70%.

In addition to the oxidizing agents and carbonyl-group-containing compounds used, the cleaning compositions according to the invention can comprise one or more bleach activators. These are compounds which liberate peroxycarboxylic acids under perhydrolysis conditions. The customary bleach activators which contain O- and/or N-acyl groups are suitable. Preference is given to polyacylated alkylenediamines, in particular tetraacetylethylenediamine (TAED), acylated glycolurils, in particular tetraacetylglycoluril (TAGU), acylated triazine derivatives, in particular 1,5-diacetyl-2,4-dioxohexahydro-1,3,5-triazine (DADHT), acylated phenolsulfonates, in particular nonanoyl or isononanoyl oxybenzenesulfonate (NOBS or ISONOBS), acylated polyhydric alcohols, in particular triacetin, ethylene glycol diacetate and 2,5-diacetoxy-2,5-dihydrofuran, and acetylated sorbitol and mannitol, and acylated sugar derivatives, in particular pentaacetylglucose (PAG), pentaacetylfructose, tetraacetylxylose and octaacetyllactose. Further possible activators are mentioned in WO 98/11880. The combinations of conventional bleach activators known from DE-44 43 177 can also be used.

A further important group of bleach activators are nitrile-group-containing compounds. These include cyanamide and derivatives thereof, such as N-cyanopiperidine, N-cyanopyrrolidine, N-cyanomorpholine and cyanopyridine. A particularly preferred class of activators are open-chain or cyclic nitrile quats, as are described, for example, in EP-A-303 520, EP-A-464 880 and WO 98/23602. Particular preference is given to trimethylamnioniumacetonitrile and morpholinomethylammonium-acetonitrile. The activators used can be used in concentrations of 0.01-25%, preferably 0.5-15%.

Moreover, the addition of small amounts of known bleach stabilizers, such as, for example, of phosphonates or aminocarboxylates, can be advantageous.

In addition to the components already mentioned, the cleaning compositions according to the invention comprise the customary constituents as described at the outset. As gas-liberating system, the cleaning composition can comprise a combination of an alkali metal carbonate or bicarbonate with an inorganic or organic acid. Alkali metal carbonates or bicarbonates which may be used are sodium carbonate. potassium carbonate, sodium bicarbonate, sodium sesquicarbonate, potassium bicarbonate or mixtures of said substances. The following compounds may be used as acid components: sulfamidic acid, acidic salts of phosphoric acid and of sulfuric acid. The organic acid which may be used is citric acid, malic acid, maleic acid or fumaric acid. When water is added, the mixture of acid and base leads to the formation of gas bubbles which aid the cleaning action of the oxidizing agents. The carbonate or bicarbonate component is used in concentrations of from 5 to 70%, preferably from 20 to 60%, and the acid component is used in concentrations of from 5 to 50%, preferably from 10 to 40%.

In addition, it may be useful for the cleaning composition to comprise further components in order to increase the cleaning action. These include, for example, surface-active substances, such as anionic, nonionic or cationic surfactants, complexing agents, enzymes, flavorings and aromas, dyes, preservatives etc.

Typical components of such cleaning compositions and their use concentrations are given in the table below:

| | | |
|---|---|---|
| Sodium perborate monohydrate | 15-30% | by wt. |
| Potassium monopersulfate | 20-40% | by wt. |
| Sodium hydrogencarbonate | 10-30% | by wt. |
| Sodium carbonate | 0-10% | by wt. |
| Sodium sulfate | 0-10% | by wt. |
| Citric acid or citrate salts or the like | 5-20% | by wt. |
| Tetraacetylethylenediamine | 0.3-3% | by wt. |
| Organic phosphonic acid or salts thereof | 0.5-3% | by wt. |
| Polyethylene glycol 20 000 | 1-4.5% | by wt. |
| Polyvinylpyrrolidone | 0-3% | by wt. |
| Silicon dioxide (Aerosil 200/300) | 0.5-1.5% | by wt. |
| Sodium dodecylbenzenesulfonate ricinyl | 0.2-1.5% | by wt. |
| monoethanolamide sulfosuccinate, disodium salt | | |
| or similarly acting surfactants | | |
| Hydrogenated triglycerides | 0-2% | by wt. |
| Fatty alcohol polyglycol ethers | 0.5-2% | by wt. |
| Preservatives, simultaneously stabilizer | 0-2% | by wt. |
| Peppermint powder or other aroma substances | 1-2% | by wt. |
| (e.g. eucalyptus) | | |
| Dye system | 0.05-0.35% | by wt. |

### EXAMPLES

A cleaning tablet in accordance with the invention is described below by reference to a specific working example.

| | | |
|---|---|---|
| 30% | by wt. | sodium perborate monohydrate |
| 20% | by wt. | potassium monopersulfate |
| 20% | by wt. | sodium hydrogencarbonate |
| 5% | by wt. | sodium carbonate |
| 4% | by wt. | sodium sulfate |
| 7% | by wt. | citric acid, sodium salt |
| 1.5% | by wt. | dipyridyl ketone |
| 1.5% | by wt. | organic phosphoric acids or salts thereof |
| 4% | by wt. | polyethylene glycol 20 000 |
| 1.5% | by wt. | polyvinylpyrrolidone |
| 1.5% | by wt. | Aerosil ® 200/300 |
| 0.75% | by wt. | sodium dodecylbenzenesulfonate |
| 0.5% | by wt. | hydrogenated triglycerides |
| 1% | by wt. | fatty alcohol polyglycol ethers |
| 1% | by wt. | preservatives |
| 0.5% | by wt. | peppermint powder and |
| 0.25% | by wt. | Indigotin ® L-Blue 2 and Chinolingelb ® |
| | | L-Yellow 3, |

were compressed as powders or in granulated form using known techniques to give a cleaning tablet. Following dissolution at a cleaning liquor temperature of 30° C., the resulting cleaning tablet exhibited a pH in the cleaning liquor of 9.0, and the cleaning process, which could be visually monitored throughout its entire course by a corresponding color change, was complete after approximately 10 minutes.

### EXAMPLE 2

Bleaching Tests

The improvement in the bleaching performance of the formulations according to the invention was demonstrated by adding various carbonyl-group-containing compounds to commercially available denture cleaners. The commercially available denture cleaners used were “Die Blauen” from Kukident® (Reckitt and Colman AG) and “Blend-a-dent 2 Phasen” (Procter & Gamble). The bleach test fabric used was tea on cotton (WFK, Krefeld).

Four bleach test fabric cloths were each added to 200 ml of water (38° C.). One tablet of the commercially available denture cleaner and 0.1 g of a carbonyl-group-containing compound were then added. Reaction time: 10 minutes. The bleach test fabric was then rinsed 2x with water and dried. The degree of whiteness of the fabric was determined using an Elrepho 3000 and compared with the degree of whiteness of the untreated test soiling.

Tests with “Die Blauen” from Kukident

Reflectance value

| | |
|---|---|
| Denture cleaner without additive | +4.1 |
| Denture cleaner with dipyridyl ketone additive | +5.4 |
| Denture cleaner with N-acetyl-4-piperidone additive | +6.3 |
| Denture cleaner with diethylaminoacetone additive | +11.0 |

Experiments with “Blend-a-dent 2 Phasen” (Procter & Gamble)

Reflectance value

| | |
|---|---|
| Denture cleaner without additive | +7.1 |
| Denture cleaner with dipyridyl ketone additive | +8.1 |
| Denture cleaner with N-acetyl-4-piperidone additive | +9.4 |
| Denture cleaner with diethylaminoacetone additive | +13.2 |

In both series of experiments, the results show a significant increase in the bleaching performance, brought about by the increase of the carbonyl-group-containing compounds.

1. A cleaning composition for dental prostheses, which comprises a) at least one oxidizing agent and b) at least one carbonyl-group-containing organic compound which forms a dioxirane structure in aqueous solution by reaction with the oxidizing agent.

2. The cleaning composition as claimed in claim 1, wherein the carbonyl-group-containing compound has a ketone structure.

3. The cleaning composition as claimed in claim 1, wherein the carbonyl-group-containing organic compound corresponds to the formulae 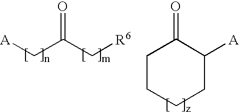
where A is a group of the formulae —NR³R⁴ or —⊕NR³R⁴R⁶; wherein R³, R⁴, R⁵ and R⁶ are C₁-C₂₂-alkyl; C₂-C₂₂-alkenyl or aryl, n is a number from 1 to 11; m is a number from 0 to 12; z is a number from 0 to 3; and A is an anion.

4. The cleaning composition as claimed in claim 1, which comprises alkali metal or ammonium peroxosulfates and/or alkali metal perborates and/or alkali metal percarbonates.

5. The cleaning composition as claimed in claim 1, which additionally comprises one or more bleach activators from the group of N-acyl or O-acyl compounds.

6. The cleaning composition as claimed in claim 1, which additionally comprises one or more bleach activators with a nitrile group.
